(19) United States
(12) Patent Application Publication
(10) Pub. No.: US 2023/0386605 A1
(43) Pub. Date: Nov. 30, 2023

(54) PREDICTING METHOD OF TRANSCRIPTION FACTOR BINDING SITES BASED ON WEIGHTED MULTI-GRANULARITY SCANNING

(71) Applicant: Shanghai Institute of Technology, Shanghai (CN)

(72) Inventors: Zhendong Liu, Shanghai (CN); Xiangdong Mao, Shanghai (CN); Yunxiang Liu, Shanghai (CN); Dongyan Li, Shandong (CN); Yu Zhang, Shanghai (CN); Tao Lin, Shanghai (CN)

(73) Assignee: Shanghai Institute of Technology, Shanghai (CN)

(21) Appl. No.: 18/305,365

(22) Filed: Apr. 23, 2023

(30) Foreign Application Priority Data
May 18, 2022 (CN) 202210535743.3

(51) Int. Cl. G16B 15/30; G16B 20/30; G16B 40/20

(52) U.S. Cl. CPC G16B 15/30; G16B 40/20; G16B 20/30

(57) ABSTRACT

The present discloses a predicting method of transcription factor binding sites based on weighted multi-granularity scanning, which belongs to the field of site prediction. The method comprises: using an inverse sequence, a complementary sequence and a complementary inverse sequence to augment an initial data set; carrying out feature representation on the DNA sequence by combining one-hot coding and multi-base feature coding; dividing a training set and a test set; calculating weight vectors of the features; carrying out weighted multi-granularity scanning; performing model training through a cascade forest to obtain a classification prediction model of transcription factor binding sites; inputting the test set into the classification prediction model to obtain a classification prediction result; and constructing an evaluation index to evaluate the performance of the method. The method overcomes only paying attention to the single-base features, long training time, and low prediction accuracy, which has high robustness and portability.

### CROSS-REFERENCE TO RELATED APPLICATION

This application claims the priority benefit of China application serial no. 202210535743 .3, filed on May 18, 2022. The entirety of the above-mentioned patent application is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Jun. 8, 2023, is named 133582_sequencing-listing and is 5,669 bytes in size.

### TECHNICAL FIELD

The present disclosure belongs to the field of site prediction, and mainly relates to a predicting method of transcription factor binding sites, in particular to a predicting method of transcription factor binding sites based on weighted multi-granularity scanning.

### BACKGROUND

In eukaryotes, gene expression is regulated by many regulatory factors. The regulation and control of genes in organisms is referred to as gene expression regulation. The regulation of gene expression has a far-reaching influence on adaptation to environmental changes and self-regulation of organisms. In eukaryotes, both the time of transcription and the rate of the transcription process can control gene expression, so that transcription regulation is closely related to gene expression regulation. Transcription factors, as a special DNA binding protein, can bind to a DNA template chain, and then regulate the transcription process. Transcription factors participate in different biological processes at all stages of life activities. The processes such as proliferation, growth, differentiation and apoptosis of cells are inseparable from the regulation effect of transcription factors. The abnormal function of transcription factors will lead to abnormal life activities, and then lead to a variety of diseases. For example, common nervous system diseases, coronary heart disease, diabetes, hypertension and even cancer are closely related to the changes of transcription factors.

Transcription factor binding sites are sites on DNA sequences that bind to transcription factors, and most of the sites are located on promoters upstream of DNA sequences. The study of transcription factor binding sites is helpful to study a series of diseases caused by site mutation. In some cancer treatments, transcription factor binding sites are also commonly used as effective drug targets, which is of great significance for research and development and innovation of drugs. At present, the predicting method of transcription factor binding sites generally have the defects that the prediction accuracy is unsatisfactory, or the accuracy is high, but the prediction experiment takes a long time, and the accuracy is unsatisfactory for a small data set, so that the current site prediction demand cannot be satisfied. Therefore, the existing methods need to be innovated.

### SUMMARY

Aiming at the defects of the existing predicting method of transcription factor binding sites, the present disclosure provides a predicting method of transcription factor binding sites based on weighted multi-granularity scanning TF_DF. TF_DF uses the combined feature representation method to better characterize the potential features of DNA sequences, and combines the weighted multi-granularity scanning method and the cascading forest technology to improve the accuracy of prediction results, so that the model pays more attention to those important features during training. The purpose is to solve the problems that the prediction accuracy not high and the model training time is long in the current predicting method of transcription factor binding sites.

The method comprises the following steps:
(1) carrying out data augmentation on an initial data set D={D₁, D₂, . . . , Dₙ}, Dᵢ={xₖ, yₖ}(1≤k≤n) of transcription factor binding sites, where xₖ represents a DNA sequence fragment and yₖ represents whether the DNA sequence fragment is a binding site, taking the value as a binding site or a non-binding site, calculating an inverse sequence, a complementary sequence and a complementary inverse sequence of each piece of data, expanding the number of data sets to four times the original number to obtain a data sets D*={D₁, D₂, . . . , D₄ₙ}, Dᵢ, ={xₖ, yₖ}(1≤k′≤4n), and randomly mixing positive and negative samples in the data set D*;
(2) carrying out one-hot coding on each piece of DNA sequence data in the data set D* with the formula

$$\{ \begin{matrix}1000 , A \\ 0001 , T \\ 0100 , C \\ 0010 , G\end{matrix}$$

to obtain a feature vector F₁, and then combining with multi-base feature coding for feature representation to obtain a feature vector F₂, splicing the feature vectors F₁ and F₂ to obtain a combined feature representation F, and encoding the result category with the formula

$$\{ \begin{matrix}1 , binding site \\ 0 , non - binding site\end{matrix} ;$$

(3) dividing the data set D* after feature representation in step (2) according to the ratio Q:R of the number of samples in the training set to the number of samples in the test set to obtain a training set Dₜᵣₐᵢₙ and a test set Dₜₑₛₜ, where Q is the number of samples in the training set in the data set D* and R is the number of samples in the test set in the data set D*; Q has a value in the range of 2-5, and R has a value of 1;
(4) using T decision trees to calculate a weight vector W=(W₁, W₂ . . . Wᵢ . . . W_{d})(1≤i≤d) for the training set Dₜᵣₐᵢₙ, where d is the length of the feature, and the specific calculation formula is as follows:

$${W}_{i} = \frac{{Score}_{i}}{{\sum }_{i = 1}^{d} {Score}_{i}}$$

where d is the total number of features, Scoreᵢ is the importance score of the i-th column of features in the weight vector W, and the specific calculation formula is as follows:

Scoreᵢ=Σₜ₌₁^{T}Score_{node}(t)

where Score_{node}(t) is the importance score of the t-th decision tree node, and the specific calculation formula is as follows:

Score_{node}=G_{node}G_{node,0} −G_{node,1}

where G_{node,0} and G_{node,1} represent the Gini index of the nodes belonging to category 0 under the node branch and the Gini index of the nodes belonging to category 1 under the node branch, respectively;

G_{node} is the Gini index of each node, and the specific formula is as follows:

$${G}_{node} = 1 - {( \frac{{N}_{node , 0}}{N} )}^{2} - {( \frac{{N}_{node , 1}}{N} )}^{2}$$

where N is the number of samples in the training set Dₜᵣₐᵢₙ, N_{node,0} is the number of nodes belonging to category 0, and N_{node,1} is the number of nodes belonging to category 1;
(5) carrying out weighted multi-granularity scanning on the feature F of each sample in the training set Dₜᵣₐᵢₙ, in which the specific steps are as follows: using a sliding window with a length of μ to slide with a step length of L on the feature vector F and the weight vector W with a length of d, respectively, and extracting the feature vectors in the window separately to obtain fᵤ and wᵤ with a length of μ, where u is the number of times the sliding window slides, and u has a value in the range of 1≤u≤d−μ+1;
according to the formula F′ᵤ=fᵤ*wᵤ^{T}, calculating the features of the weighted multi-granularity scanning, where wᵤ^{T} is the transposition of vector wᵤ; sending the feature F′ᵤ into a completely random forest A and an ordinary random forest B to obtain F′_{Au} and F′_{Bu} , respectively, and finally, splicing F′_{Au} and F′_{Bu} to obtain feature F*;
(6) inputting F* into the cascade forest, carrying out the model training to obtain a classification prediction model of transcription factor binding sites, inputting the test set Dt es t into the classification prediction model, and outputting a result of 1 or 0; in which 1 indicates that the DNA sequence is a transcription factor binding site, and 0 indicates that the DNA sequence is a non-transcription factor binding site.

Preferably, in the multi-base feature coding method, the length L of the feature column is obtained according to the formula L=4^{m}, where m is the length of the base in the multi-base, m has a value of 3, bases A, T, C and G form a sequence set C with a length of 3 bp: {'AAA', ‘AAT’, ‘AAG’, ‘AAC’, ‘ATA’, ‘ATT’, ‘ATG’, ‘ATC’, ‘AGA’, ‘AGT’, ‘AGG’, ‘AGC’, ‘ACA’, ‘ACT’, ‘ACG’, ‘ACC’, ‘TAA’, ‘TAT’, ‘TAG’, ‘TAC’, ‘TTA’, ‘TTT’, ‘TTG’, ‘TTC’, ‘TGA’, ‘TGT’, ‘TGG’, ‘TGC’, ‘TCA’, ‘TCT’, ‘TCG’, ‘TCC’, ‘GAA’, ‘GAT’, ‘GAG’, ‘GAC’, ‘GTA’, ‘GTT’, ‘GTG’, ‘GTC’, ‘GGA’, ‘GGT’, ‘GGG’, ‘GGC’, ‘GCA’, ‘GCT’, ‘GCG’, ‘GCC’, ‘CAA’, ‘CAT’, ‘CAG’, ‘CAC’, ‘CTA’, ‘CTT’, ‘CTG’, ‘CTC’, ‘CGA’, ‘CGT’, ‘CGG’, ‘CGC’, ‘CCA’, ‘CCT’, ‘CCG’, ‘CCC’}, each element in set C is set as a feature column, there are 64 feature columns in total, and its element is the feature name of the feature column;
the calculation method of the feature vector F₂ is as follows: from the beginning end of the DNA sequence sample, a window with a step size of 1 and a length of 3 bp slides on the DNA sequence sample and extracts features, and the feature column corresponding to the sequence in the window has a value of 1 until the end of the DNA sequence sample, that is, the length of the feature vector F₂ is 64.

Preferably, in step (3), Q has a value of 4, and R has a value of 1.

Preferably, in step (4), T has a value of 462, and the maximum depth of the tree is 11.

Preferably, in step (5), μ has a value of 50, and L has a value of 1.

### BRIEF DESCRIPTION OF THE DRAWINGS

FIG. 1 is a flowchart of a predicting method of transcription factor binding sites based on weighted multi-granularity scanning.

FIG. 2 is a schematic diagram of DNA sequence expansion to construct a data set.

FIG. 3 is a schematic diagram of one-hot coding rules of DNA sequences.

FIG. 4 is a schematic diagram of the transformation of DNA sequence data into the feature representation combining one-hot coding and multi-base feature coding.

FIG. 5 is a diagram showing the calculation results of the weight of DNA sequence features.

FIG. 6 is a flowchart of a weighted multi-granularity scanning method.

FIG. 7 is a comparison diagram showing the results of the category accuracy of predicting transcription factor binding sites by combining a feature representation method and a single-base feature representation method.

### DETAILED DESCRIPTION OF THE EMBODIMENTS

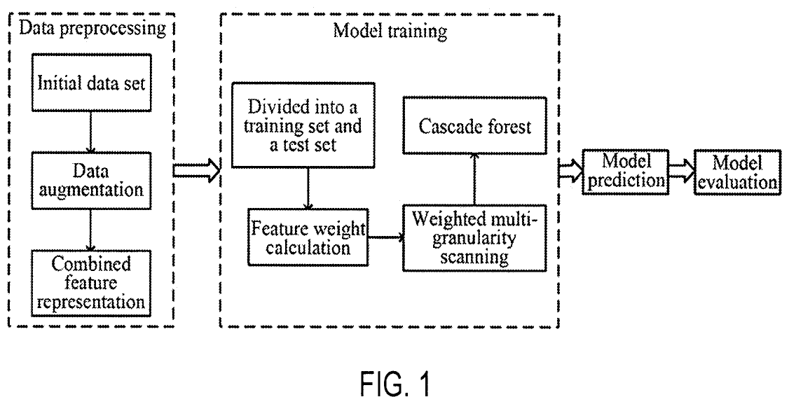

In order to clearly illustrate the technical scheme of the present disclosure, the present disclosure will be described in conjunction with FIG. 1 to FIG. 7 and examples. The examples here are only used for explaining the present disclosure, rather than limit the present disclosure.

It should be pointed out that the following detailed description is exemplary and is intended to provide further explanation of the present disclosure. Unless otherwise specified, all technical and scientific terms used herein have the same meaning as commonly understood by those skilled in the art to which the present disclosure belongs.

FIG. 1 shows the process steps of predicting transcription factor binding sites using the TF_DF method. In the data preprocessing stage, data augmentation and feature extraction are carried out on the initial data set, and the model is constructed by using the processed features. Based on the weighted multi-granularity scanning technology, the model is trained by combining the cascade forest technology, and the performance of the model is verified by using the test set. It can be explained that the method is also applicable to other DNA binding sites and genetic elements based on sequence features. The data set selected in the embodiment is the data set of transcription factor SP1 binding sites of human chromosome 1.

The input file of the TF_DF method contains a CSV-type file. Raw data.csv file contains 1200 positive samples and 1200 negative samples of the transcription factor SP1 binding sits of human chromosome 1, which is the original data set D. Each piece of data contains a DNA sequence with a length of 14 bases and its corresponding categories (binding sites and non-binding sites), and the initial data is preprocessed on the basis of the data set. The output file of the TF_DF method contains a CSV-type file and an output-type file. The file sequence feature.csv is the data set D* obtained by data preprocessing. The file TF_classification.output is the prediction category of each site in the test set output by using the TF_DF method. The output of the TF_DF method is whether each DNA sequence predicted by the method is a transcription factor binding site.

The TF_DF predicting method can be specifically divided into the following steps.

### 1. Data Preprocessing

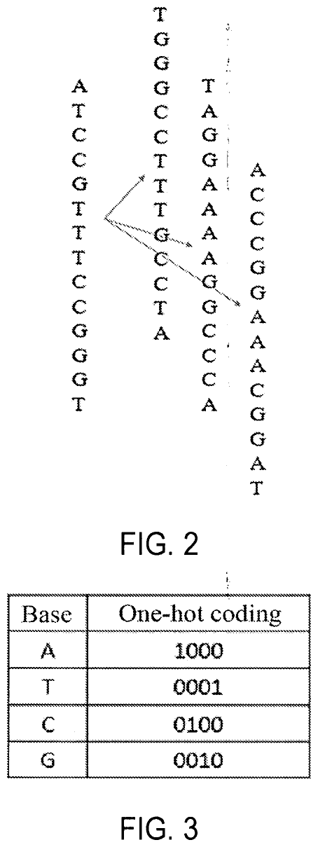

In this embodiment, the data set D={D₁, D₂, . . . , Dₙ} of the transcription factor SP1 binding sites of human chromosome 1 is preprocessed. Taking the small amount of data into account, it is necessary to carry out data augmentation on the data set first. According to the sequence features of DNA binding sites, the inverse sequence, the complementary sequence and the complementary inverse sequence of each DNA sequence are found, and the number of positive samples and the number of negative samples both expand to 4800 (FIG. 2). The positive and negative samples are randomly mixed. Then, one-hot coding is carried out on each piece of DNA sequence data in the data set D* with the formula

$$\{ \begin{matrix}1000 , A \\ 0001 , T \\ 01 \; 00 , C \\ 0010 , G\end{matrix}$$

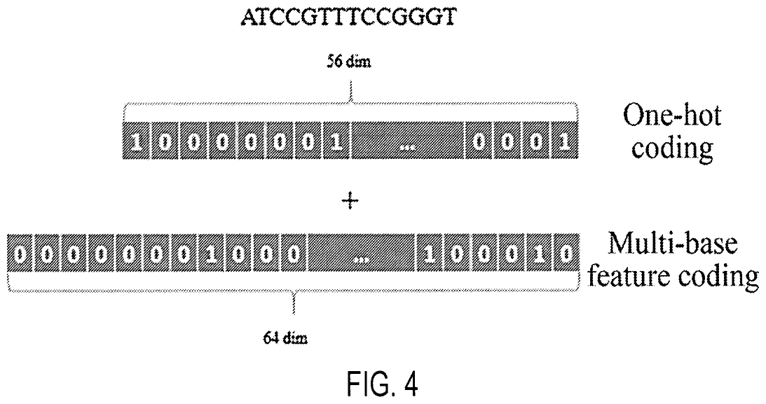

to obtain a feature vector F₁ (FIG. 3). Finally, the length of the sequence fragment is set to 3 bp in the multi-base feature coding, that is, the length of the sequence set C with the length of 3 bp consisting of bases A, T, C and G is 64 (64 feature columns). If each piece of data contains a certain sequence feature, the feature column corresponding to the data is denoted as “1” to form a feature vector F₂. Finally, the feature F of each piece of data obtained is a combination of one-hot coding and multi-base feature coding, that is, the splicing of feature vector F₁ and feature vector F₂ (FIG. 4). Data preprocessing operation (taking data {‘(SEQ ID NO:4) ATCCGTTTCCGGGT’, ‘binding site’} as an example):
(1) three pieces of data augmented according to the inverse sequence, the complementary sequence and the complementary inverse sequence of the DNA sequence are {‘(SEQ ID NO:1) TGGGCCTTTGCCTA’, ‘binding site’}, {‘(SEQ ID NO:2) TAGGAAAAGGCCCA’, ‘binding site’}, {‘(SEQ ID NO:3) ACCCGGAAACGGAT’, ‘binding site’};
(2) the data {‘(SEQ ID NO:4) ATCCGTTTCCGGGT’, ‘binding site’} is taken as an example to show an example of feature extraction, and one-hot coding is carried out on the DNA sequence data to obtain the feature vector Fl (1, 0, 0, 0, 0, 0, 0, 1, 0, 1, 0, 0, 0, 1, 0, 0, 0, 0, 1, 0, 0, 0, 1, 0, 0, 0, 1, 0, 0, 0, 1, 0, 1, 0, 0, 0, 1, 0, 0, 0, 0, 1, 0, 0, 0, 1, 0, 0, 0, 1, 0, 0, 0, 0, 1);
(3) feature representation is carried out on the DNA sequence by combining with multi-base feature coding to obtain the feature vector F₂ (0, 0, 0, 0, 0, 0, 0, 1, 0, 0, 0, 0, 0, 0, 0, 0, 0, 0, 0, 0, 0, 1, 0, 1, 0, 0, 0, 0, 0, 0, 0, 1, 0, 0, 0, 0, 0, 1, 0, 0, 0, 1, 1, 0, 0, 0, 0, 0, 0, 0, 0, 0, 0, 0, 0, 0, 1, 1, 0, 0, 0, 1, 0);
(4) the feature vector F₁ and the feature vector F₂ are spliced to obtain the feature vector F (1, 0, 0, 0, 0, 0, 0, 1, 0, 1, 0, 0, 0, 1, 0, 0, 0, 0, 1, 0, 0, 0, 0, 1, 0, 0, 0, 1, 0, 0, 0, 1, 0, 1, 0, 0, 0, 1, 0, 0, 0, 1, 0, 0, 0, 1, 0, 0, 0, 1, 0, 0, 0, 0, 1, 0, 0, 0, 0, 0, 0, 0, 1, 0, 0, 0, 0, 0, 0, 0, 0, 0, 0, 0, 0, 0, 1, 0, 1, 0, 0, 0, 0, 0, 0, 0, 1, 0, 0, 0, 0, 0, 1, 0, 0, 0, 1, 1, 0, 0, 0, 0, 0, 0, 0, 0, 0, 0, 0, 0, 0, 0, 1, 1, 0, 0, 1, 0);
(5) the result category is encoded with the formula

$$\{ \begin{matrix}1 , binding site \\ 0 , non - binding site\end{matrix} ,$$

that is, the result category is the transcription factor binding sites.

In this embodiment, the data set D* after data preprocessing contains 4800 positive samples and 4800 negative samples, and each piece of sample data contains 120 feature items and 1 result feature category. The positive and negative samples are disrupted and mixed.

### 2. Dividing the Training Set and the Test Set

The data set D* after feature representation in step (2) is divided according to the ratio 4:1 of the number of samples in the training set to the number of samples in the test set to obtain a training set Dₜᵣₐᵢₙ and a test set Dₜₑₛₜ; the number of samples in the training set Dₜᵣₐᵢₙ and the number of samples in the test set Dₜₑₛₜ are 7680 and 1920 after the data set is divided in this example, respectively.

### 3. Feature Weight Calculation

462 decision trees are used to calculate the weight vector W of the training set Dtrain. According to the formula the Gini

$${G}_{node} = 1 - {( \frac{{N}_{node , 0}}{N} )}^{2} - {( \frac{{N}_{node , 1}}{N} )}^{2} ,$$

index G node of each node is calculated, where N is the number of samples in the training set Dₜᵣₐᵢₙ, N_{node,0} is the number of nodes belonging to category 0, and N_{node,1} is the number of nodes belonging to category 1. According to the formula Score_{node}=G_{node}−G_{node,0}−Gnode_{,1} the importance score Score_{node} of each node is calculated, where G_{node,0} and G_{node,1} represent the Gini index of the nodes belonging to category 0 under the node branch and the Gini index of the nodes belonging to category 1 under the node branch, respectively. According to the formula Scoreᵢ=Σₜ₌₁^{T}Score_{node}, the importance score Scoreᵢ of the i-th column of features is calculated, where

T is the number of decision trees. According to the formula

$${W}_{i} = \frac{{Score}_{i}}{{\sum }_{i = 1}^{d} {Score}_{i}} ,$$

the weight Wᵢ of each feature is calculated, where Scoreᵢ is the importance score of the i-th column of features, and d is the total number of features.

In this example, top 10 partial features in the weight and their corresponding weight results are as follows:

| Feature | Weight |
|---|---|
| GCG | 0.090326 |
| CGC | 0.089976 |
| CGG | 0.061189 |
| CCG | 0.059855 |
| GCC | 0.059811 |
| GGC | 0.057544 |
| CCC | 0.027319 |
| TAA | 0.023469 |
| AAT | 0.022187 |
| TTA | 0.021713 |

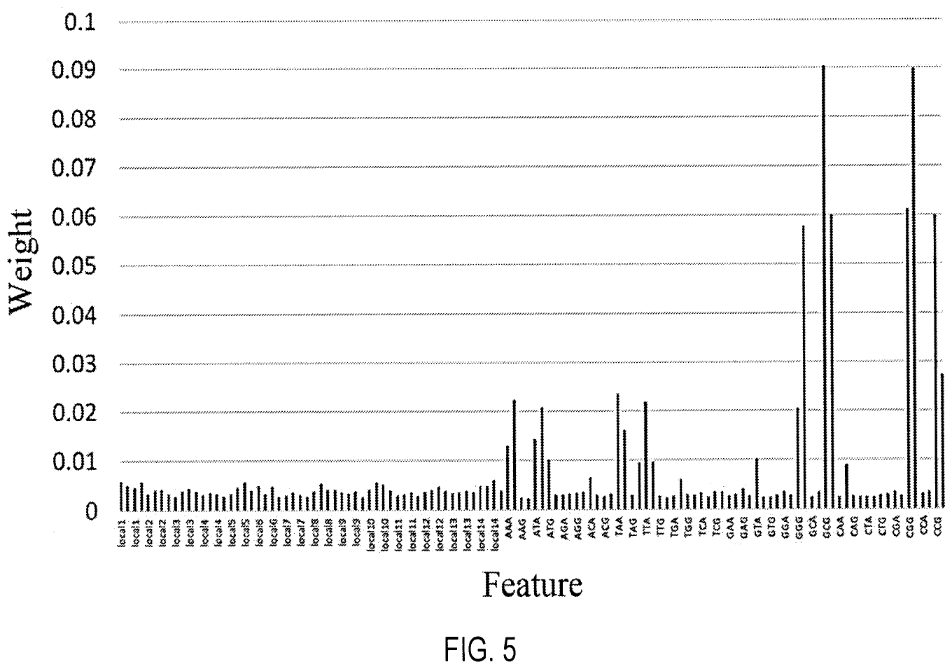

FIG. 5 shows all the features of the DNA sequence and the calculation results of its weights.

### 4. Weighted Multi-Granularity Scanning

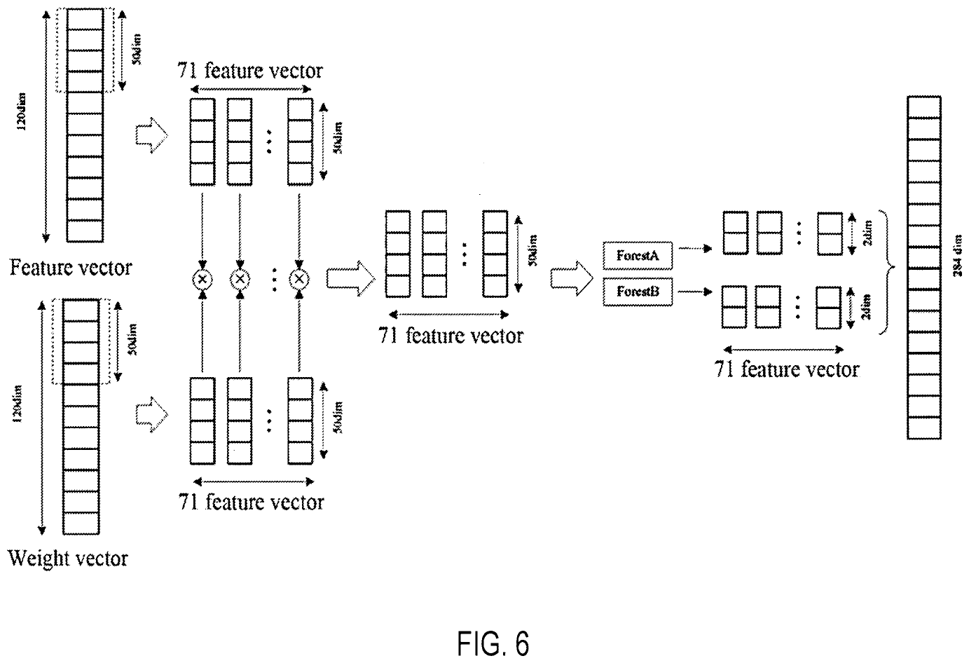

As shown in FIG. 6, weighted multi-granularity scanning is carried out on the feature F of each sample in the training set Dₜᵣₐᵢₙ, and the specific steps are as follows: using a sliding window with a length of μ to slide on the feature vector F and the weight vector W with a length of 120, respectively, to obtain fᵢ and wᵤ (1<u<120-μ+1). According to the formula F′ᵤ=fᵤ*wᵤ^{T}, the features of the weighted multi-granularity scanning is calculated, where wᵤ^{T} is the transposition of vector wᵤ. The feature F′ᵤ is sent into a completely random forest A and an ordinary random forest B to obtain F′_{Au} and F′_{Bu}, respectively. Finally, F′_{Au} and F′_{Bu} are spliced to obtain feature F*.

### 5. Prediction of Transcription Factor Binding Sites

F* is input into the cascade forest, and the model training is carried out to obtain a classification prediction model of transcription factor binding sites. The test set Dₜₑₛₜ is input into the classification prediction model to verify the performance of the model.

Take the predicted DNA sequence “(SEQ ID NO:5) GGGGCGGGGCCGGC” as an example. Then the final classification prediction result of the DNA sequence is ‘1 ’, which is the transcription factor binding site.

### 6. Evaluation of the Performance of the Method

According to five-fold cross-validation and three evaluation indexes, the performance of the method is evaluated, and the accuracy of the method and F₁ value are calculated with the formula

$$accuracy = \frac{a}{b}$$

and the formula

$${F}_{1} = \frac{2 * p * r}{p + r} ,$$

respectively, where a is the number of samples in which the predicted classification results are the same as the actual classification results, and b is the number of samples in the test set Dₜₑₛₜ. p value and r value are calculated with the formula

$$p = \frac{T P}{T P + F P}$$

and the formula

$$r = \frac{T P}{T P + F N} ,$$

respectively, where TP is the number of data points in which the predicted classification result is the transcription factor binding site and the actual classification result is the transcription factor binding sites, FP is the number of data points in which the predicted classification result is the transcription factor binding site but the actual classification result is the non-transcription factor binding site, and FN is the number of data points in which the predicted classification result is the non-transcription factor binding site but the actual classification result is the transcription factor binding site. Accuracy can be regarded as the percentage of the correct rate of the algorithm output results with the value in the range of [0,1]. The closer the accuracy is to 1, the larger the number of correctly predicted samples, and the closer the accuracy is to 0, the smaller the number of correctly classified results. When the value F₁ is higher, it can be shown that the algorithm is closer to the ideal state. The AUC value is the area surrounded by the coordinate axis under the ROC curve, which can more objectively reflect the ability of the model. Generally speaking, the higher the AUC value, the stronger the performance of the algorithm. It can be known through the above calculation formula that the accuracy, the value F₁ and the AUC of the test set Dₜₑₛₜ are 0.8943, 0.8920 and 0.9219, respectively.

The features of a single base are important for identifying TFBS in DNA sequence, and the base next to each base can be also important. In order to prove this idea, the single basic feature is compared with the features represented by combining the multi-base feature coding method on several models.

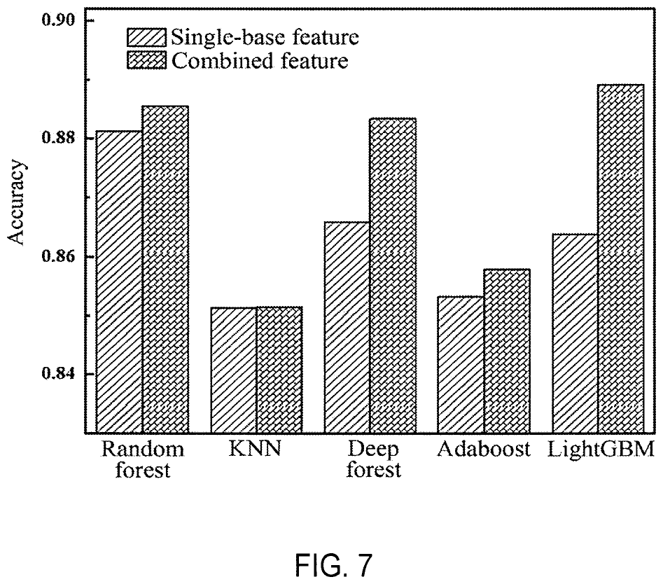

The experimental results (FIG. 7) show that in all algorithms, the accuracy of classification prediction results using combined features is better than that of classification prediction results using a single feature to varying degrees. After using Deep Forest and LightGBM algorithms, the accuracy of prediction results has been effectively improved by 1.75% and 2.54%, respectively. Therefore, it can be concluded that the combined features improve the extraction of DNA sequence features. It is thought that the combined feature representation can capture more feature information in the DNA sequence. In the experiment, the best result is obtained when the length of the feature sequence is set to 3 bp, which can be related to the fact that amino acids consist of three bases.

The data set D* is input into the TF_DF method for model training after being divided, so as to realize the high-accuracy prediction of each point in the prediction set. 15 experiments have been carried out on all the proposed classification algorithms. In order to ensure a fair comparison, the same training data and test data are used in each experiment, and the parameter settings of each model are also the same. The following table shows the average results of 15 experiments of KNN, Adaboost, Random Forest, LightGBM, Deep Forest and TF_DF method.

| Classifier | accuracy | value F₁ | AUC |
|---|---|---|---|
| Random Forest | 0.8833 | 0.8813 | 0.9158 |
| KNN | 0.8514 | 0.8551 | 0.8819 |
| Deep Forest | 0.8872 | 0.8864 | 0.9166 |
| Adaboost | 0.8579 | 0.8610 | 0.9001 |
| LightGBM | 0.8794 | 0.8785 | 0.9055 |
| TF_DF | 0.8943 | 0.8920 | 0.9219 |

In contrast, the accuracy, the F1 value and the AUC of the TF_DF method are 89.43%, 89.20% and 92.19%, respectively, which are higher than other classification algorithms to varying degrees. This shows that the TF_DF method has higher prediction ability. Compared with the experimental results, it can be concluded that the TF_DF method designed by the present disclosure improves the accuracy, ability and performance of the classifier. That is to say, the TF_DF method is better in effect than previous classification algorithms in the field of classification and prediction of transcription factor binding sites.

Compared with the method in the prior art, the method has the following beneficial effects.

The TF_DF method realizes highly accurate prediction of transcription factor binding sites, especially the site prediction for small data sets. The method abandons the idea of a single-base feature and combines multi-base feature coding to extract the features of each base context, which improves the accuracy rate of classification and prediction results. At the same time, based on the idea of different importance of features, the multi-granularity scanning is optimized to obtain better performance, and the cascade forest is used to train and predict the model. Compared with the existing predicting method of transcription factor binding sites, the present disclosure has higher efficiency and accuracy, and has better robustness and portability.

Finally, it should be explained that the above is only a preferred embodiment of the present disclosure, rather than limit the present disclosure. Although the present disclosure has been described in detail with reference to the aforementioned embodiments, those skilled in the art can still modify the technical scheme described in the aforementioned embodiments or to replace some technical features equivalently. Any modification, equivalent replacement, improvement, etc. made within the spirit and principle of the present disclosure should be included in the protection scope of the present disclosure.

What is claimed is:

1. A predicting method of transcription factor binding sites based on weighted multi-granularity scanning, comprising the following steps:
(1) carrying out data augmentation on an initial data set D={D₁, D₂, . . . , Dₙ}, Dᵢ={xₖ, yₖ}(1≤k≤n) of transcription factor binding sites, where xₖ represents a DNA sequence fragment and y k represents whether the DNA sequence fragment is a binding site, taking the value as a binding site or a non-binding site, calculating an inverse sequence, a complementary sequence and a complementary inverse sequence of each piece of data, expanding the number of data sets to four times the original number to obtain a data sets D*={D₁, D₂, . . . , D₄ₙ}, Dᵢ, ={xₖ, yₖ}(1≤k′≤4n), and randomly mixing positive and negative samples in the data set D*;
(2) carrying out one-hot coding on each piece of DNA sequence data in the data set D* with the formula
$$\{ \begin{matrix}1000 , A \\ 0001 , T \\ 0100 , C \\ 0010 , G\end{matrix}$$
to obtain a feature vector F₁, and then combining with multi-base feature coding for feature representation to obtain a feature vector F₁, splicing the feature vectors F₁ and F₂ to obtain a combined feature representation F, and encoding the result category with the formula
$$\{ \begin{matrix}1 , binding site \\ 0 , non - binding site\end{matrix} ;$$
(3) dividing the data set D* after feature representation in step (2) according to the ratio Q:R of the number of samples in the training set to the number of samples in the test set to obtain a training set Dₜᵣₐᵢₙ and a test set Dₜₑₛₜ, where Q is the number of samples in the training set in the data set D* and R is the number of samples in the test set in the data set D*; Q has a value in the range of 2-5, and R has a value of 1;
(4) using T decision trees to calculate a weight vector W=(W₁, W₂ . . . Wᵢ . . . W_{d})(1≤i≤d)for the training set Dₜᵣₐᵢₙ, where d is the length of the feature, and the specific calculation formula is as follows:
$${W}_{i} = \frac{{Score}_{i}}{{\sum }_{i = 1}^{d} S c o r {e}_{i}}$$
where d is the total number of features, Scoreᵢ is the importance score of the i-th column of features in the weight vector W, and the specific calculation formula is as follows:
$${Score}_{i} = {\sum }_{t = 1}^{T} {Score}_{node} ( t )$$
where Score_{node}(t) is the importance score of the t-th decision tree node, and the specific calculation formula is as follows:
Score_{node}=G_{node}G_{node,0} −G_{node,1}
where G_{node,0} and G_{node,1} represent the Gini index of the nodes belonging to category 0 under the node branch and the Gini index of the nodes belonging to category 1 under the node branch, respectively;
G_{node} is the Gini index of each node, and the specific formula is as follows:
$${G}_{node} = 1 - {( \frac{{N}_{node , 0}}{N} )}^{2} - {( \frac{{N}_{node , 1}}{N} )}^{2}$$
where N is the number of samples in the training set Dₜᵣₐᵢₙ, Nnode,o is the number of nodes belonging to category 0, and N_{node,1} is the number of nodes belonging to category 1;
(5) carrying out weighted multi-granularity scanning on the feature F of each sample in the training set Dₜᵣₐᵢₙ, in which the specific steps are as follows: using a sliding window with a length of μ to slide with a step length of L on the feature vector F and the weight vector W with a length of d, respectively, and extracting the feature vectors in the window separately to obtain fᵤ and wᵤ with a length of μ, where u is the number of times the sliding window slides, and u has a value in the range of 1≤u≤d−μ+1;
according to the formula F′ᵤ=fᵤ*wᵤ^{T}, calculating the features of the weighted multi-granularity scanning, where wᵤ^{T} is the transposition of vector wᵤ; sending the feature F′ᵤ into a completely random forest A and an ordinary random forest B to obtain F′_{Au} and F′_{Bu}, respectively, and finally, splicing F′_{Au} and F′_{Bu} to obtain feature F*;
(6) inputting F* into the cascade forest, carrying out the model training to obtain a classification prediction model of transcription factor binding sites, inputting the test set D test into the classification prediction model, and outputting a result of 1 or 0; in which 1 indicates that the DNA sequence is a transcription factor binding site, and 0 indicates that the DNA sequence is a non-transcription factor binding site.

2. The predicting method of transcription factor binding sites based on weighted multi-granularity scanning according to claim 1, wherein in the multi-base feature coding method, the length L of the feature column is obtained according to the formula L=4^{m}, where m is the length of the base in the multi-base, m has a value of 3, bases A, T, C and G form a sequence set C with a length of 3 bp: {'AAA', ‘AAT’, ‘AAG’, ‘AAC’, ‘ATA’, ‘ATT’, ‘ATG’, ‘ATC’, ‘AGA’, ‘AGT’, ‘AGG’, ‘AGC’, ‘ACA’, ‘ACT’, ‘ACG’, ‘ACC’, ‘TAA’, ‘TAT’, ‘TAG’, ‘TAC’, ‘TTA’, ‘TTT’, ‘TTG’, ‘TTC’, ‘TGA’, ‘TGT’, ‘TGG’, ‘TGC’, ‘TCA’, ‘TCT’, ‘TCG’, ‘TCC’, ‘GAA’, ‘GAT’, ‘GAG’, ‘GAC’, ‘GTA’, ‘GTT’, ‘GTG’, ‘GTC’, ‘GGA’, ‘GGT’, ‘GGG’, ‘GGC’, ‘GCA’, ‘GCT’, ‘GCG’, ‘GCC’, ‘CAA’, ‘CAT’, ‘CAG’, ‘CAC’, ‘CTA’, ‘CTT’, ‘CTG’, ‘CTC’, ‘CGA’, ‘CGT’, ‘CGG’, ‘CGC’, ‘CCA’, ‘CCT’, ‘CCG’, ‘CCC’}, each element in set C is set as a feature column, there are 64 feature columns in total, and its element is the feature name of the feature column;
the calculation method of the feature vector F₂ is as follows: from the beginning end of the DNA sequence sample, a window with a step size of 1 and a length of 3 bp slides on the DNA sequence sample and extracts features, and the feature column corresponding to the sequence in the window has a value of 1 until the end of the DNA sequence sample, that is, the length of the feature vector F₂ is 64.

3. The predicting method of transcription factor binding sites based on weighted multi-granularity scanning according to claim 1, wherein in step (3), Q has a value of 4, and R has a value of 1.

4. The predicting method of transcription factor binding sites based on weighted multi-granularity scanning according to claim 1, wherein in step (4), T has a value of 462, and the maximum depth of the tree is 11.

5. The predicting method of transcription factor binding sites based on weighted multi-granularity scanning according to claim 1, wherein in step (5), μ has a value of 50, and L has a value of 1.


SEQUENCE LISTING (5 sequences)

SEQ ID NO: 1 - DNA, 14 bp, synthetic construct
  source 1..14: mol_type = other DNA
tgggcctttgccta

SEQ ID NO: 2 - DNA, 14 bp, synthetic construct
  source 1..14: mol_type = other DNA
taggaaaaggccca

SEQ ID NO: 3 - DNA, 14 bp, synthetic construct
  source 1..14: mol_type = other DNA
acccggaaacggat

SEQ ID NO: 4 - DNA, 14 bp, synthetic construct
  source 1..14: mol_type = other DNA
atccgtttccgggt

SEQ ID NO: 5 - DNA, 14 bp, synthetic construct
  source 1..14: mol_type = other DNA
ggggcggggccggc